(19) United States
(12) Patent Application Publication
(10) Pub. No.: US 2005/0119627 A1
(43) Pub. Date: Jun. 2, 2005

(54) Selectively passive forward shielding medical needle device

(75) Inventor: Jamieson Crawford, New York, NY (US)

Correspondence Address: DAVID W. HIGHET, VP AND CHIEF IP COUNSEL;BECTON DICKINSON AND COMPANY, [THE WEBB LAW FIRM], FRANKLIN LAKES, NJ, 07414-1880, US

(73) Assignee: Becton, Dickinson and Company

(21) Appl. No.: 10/725,041

(22) Filed: Dec. 1, 2003

(51) Int. Cl. A61M 5/00

(52) U.S. Cl. 604/263; 604/110

(57) ABSTRACT

A shieldable needle device for shielding used needle cannulas. The device generally includes a hub housing and an elongated shield housing. A needle cannula having a distal puncture tip extends from the hub housing, with at least a portion of the needle cannula extending through the passageway of the shield housing. A biasing member acts on the shield housing to bias the shield housing from a first biased position adjacent the hub housing toward a second position covering the distal puncture tip of the needle cannula. An engagement mechanism extends dorsally from the hub housing and is in releasable engagement with a portion of the shield housing for releasably retaining the shield housing in the first biased position adjacent the hub housing. Activation of the engagement mechanism to shield the needle can be effectively accomplished during removal of the needle device from a patient, thereby providing for a passive activation of the device during normal use.

### BACKGROUND OF THE INVENTION

1. Field of the Invention

The present invention relates to a medical device for safe and convenient handling of used needle cannulas. More particularly, the present invention relates to a medical needle device that is adapted to automatically shield a needle cannula during normal use in a medical procedure.

2. Description of Related Art

Blood collection sets or intravenous (IV) infusion sets typically include a needle cannula having a proximal end, a distal end with a puncture tip, and a lumen extending therebetween. The proximal end of the needle cannula is mounted to a plastic hub having a central passage that communicates with the lumen in the needle cannula. A thin flexible thermoplastic tube is connected to the hub and communicates with the lumen in the needle cannula. The end of the plastic tube remote from the needle cannula may include a fixture for connecting the needle cannula to a separate medical device, such as a holder, a blood collection tube, and the like.

In order to reduce the risk of incurring an accidental needle-stick wound, protection of used needle cannulas becomes important. With concern about infection and transmission of diseases, methods and devices to cover used needle cannulas have become important and in great demand. For example, some needle assemblies commonly employ a safety shield that may be moved into shielding engagement with a used needle cannula without risking an accidental needle stick.

U.S. Pat. No. 5,505,711 discloses a winged injector needle assembly including a needle cannula attached to a hub and a holder forming a shield portion. The hub is slidable within the shield portion. The needle assembly also includes a latching mechanism between the hub and the shield portion to maintain the hub in a forward position with the needle cannula extending from the shield portion. Upon grasping of and release of the latching mechanism, the shield portion may be moved forward to shield the needle cannula. U.S. Pat. No. 5,928,199 discloses a similar forward shielding needle assembly, which further includes a lock for locking the shield portion in the forward position shielding the needle cannula. Release of the latching mechanism is awkward due to the relation between the latching mechanism extending between the shield portion and the hub. Further, this device requires the operator to exert a substantial force to shield the needle cannula, and is prone to rotation of the needle cannula within the shield portion during the shielding operation.

U.S. Pat. Nos. 5,779,679 and 6,210,371 to Shaw disclose winged IV sets with a retracting needle assembly adapted to automatically retract a needle cannula within a safety shield upon release of a latch. The winged IV sets include a pair of latch wings associated with an outer shield that hold the needle assembly in an unretracted position, and which may be released to cause the needle assembly to be retracted rearwardly within the outer shield. A spring drives the needle assembly rearward to the retracted position within the safety shield. However, since retraction of such a device is achieved by holding the safety shield, the user does not hold the needle cannula during retraction and therefore does not maintain control over the needle cannula. Thus, if the latch is released while the needle cannula is inserted in a patient or during withdrawal from the patient, the spring force may cause an undesirable partial or full retraction of the needle cannula, instead of merely passively activating the device to allow for complete retraction upon full removal from the patient.

In view of the foregoing, there is a continuing need for a shielding medical needle device adapted to shield a used needle cannula once a medical procedure is completed.

### SUMMARY OF THE INVENTION

The present invention relates generally to a safety needle system in the form of a shielding medical needle device for shielding used needle cannulas. The shielding medical needle device generally includes a hub housing and an elongated shield housing. A needle cannula having a distal puncture tip extends from the hub housing, with at least a portion of the needle cannula extending through the passageway of the shield housing. A biasing member such as a compression spring acts on the shield housing to bias the shield housing from a first biased position adjacent the hub housing toward a second position covering the distal puncture tip of the needle cannula. An engagement mechanism extends dorsally from the hub housing and is in releasable engagement with a portion of the shield housing for releasably retaining the shield housing in the first biased position adjacent the hub housing. Activation of the engagement mechanism releases the engagement mechanism from engagement with the shield housing. As such, the shield housing is released from the biased position, with the biasing member causing the shield housing to move toward the second position. Since the engagement mechanism extends dorsally from the device, activation of the device to shield the needle can be effectively accomplished during removal of the needle device from a patient, thereby providing for a passive activation of the device during normal use.

Desirably, the engagement mechanism is comprised of a first member extending dorsally from the hub housing, and a second member extending from a portion of the first member moveable with respect to the first member, such as through a fulcrum. The second member releasably engages a portion of the shield housing, such as through a hook element engaged within a recess or opening which extends through a latch element extending dorsally from the shield housing. Activation of the mechanism can be accomplished by movement of the second member with respect to the first member about the fulcrum, so as to release the hook element from engagement within the opening of the latch element of the shield housing.

Desirably, the needle system includes a pair of flexible wings extending from opposing lateral sides of the hub housing. In such an arrangement, the engagement mechanism extending dorsally from the hub housing desirably bisects the flexible wings extending from opposing lateral sides of the hub housing, but bending of the flexible wings toward a dorsal position does not cause activation of the engagement mechanism.

Additionally, the safety needle system may include a locking mechanism for locking the shield housing in the shielding position, such as by providing the hub housing with at least one flexible cut out portion along a wall thereof for engagement with a portion of the shield housing when the shield housing is in the second position to prevent a return movement of the shield housing to the first position.

In a further embodiment, the present invention is directed to a method for passively activating a safety needle system. The method includes providing a safety needle system comprising a hub housing including a needle cannula extending from a distal end of the hub housing toward a distal puncture tip; a shield housing covering at least a portion of the needle cannula adjacent the hub housing; a biasing element for biasing the shield housing toward a shielding position fully covering the distal puncture tip of the needle cannula; and a latch mechanism extending dorsally from the safety needle system for maintaining the shield housing in a biased state adjacent the hub housing. The method further includes inserting the safety needle system into a patient, and withdrawing the safety needle system from the patient by grasping the latch mechanism, thereby releasing the shield housing from the biased state and causing the shield housing to extend toward the shielding position. Desirably, the hub housing further includes a pair of flexible wings extending from opposing lateral sides thereof. In such an arrangement, the inserting step may include bending the flexible wings to a dorsal position for guiding the needle cannula into the patient. Such bending of the flexible wings to a dorsal position does not cause the latch mechanism to release the shield housing from the biased state. Further, the shield housing may be prevented from re-exposing the distal puncture tip of the needle cannula once the shield housing has been extended to a fully shielding position, such as through a locking mechanism.

In yet a further embodiment, the present invention is directed to a safety needle system which includes a grippable dorsal housing having distal and proximal dorsal housing portions. The proximal dorsal housing portion supports a needle, and the distal dorsal housing portion extends in an axial direction with respect to the needle from a first position adjacent the proximal housing portion to a second position covering a distal tip of the needle. A planar wing structure is integral to at least one of the proximal and/or distal dorsal housing portions, and extends in a direction which is generally normal to the grippable dorsal housing. A biasing element such as a spring extends between the distal and proximal dorsal housing portions for biasing the dorsal housing portion toward the second position. A release element, such as a release latch on the proximal dorsal housing portion in interference engagement with a recess or opening in the distal dorsal housing portion, selectively retains the distal dorsal housing portion in the first position adjacent the first housing portion against the bias of the biasing element. Activation of the release element causes the biasing element to act on the distal dorsal housing portion to move the distal dorsal housing portion to the second position.

Further details and advantages of the present invention will become apparent from the following detailed description when read in conjunction with the drawings, wherein like parts are designated with like reference numerals and lower case letters are included where necessary to identify specific embodiments of the invention.

### DESCRIPTION OF THE DRAWINGS

FIG. 1 is a perspective view of the safety needle assembly of the present invention in the form of a blood collection set, shown in a ready-for-use position with a packaging cover removed and the needle shield retracted to expose the puncture tip of the needle;

FIG. 2 is an exploded perspective view of the assembly of FIG. 1;

FIG. 3 is a perspective view of the needle assembly of FIG. 1 shown with the laterally extending wing members bent into a dorsal position for use during insertion of the needle assembly;

FIG. 4 is a perspective view of the needle assembly of FIG. 1 shown in a fully shielding position covering the puncture tip of the needle;

FIG. 5 is a side view of the needle assembly of FIG. 1 with the needle shield in a retracted position;

FIG. 6 is a side view of the needle assembly of FIG. 4 with the needle shield in a fully shielding position;

FIG. 7 is a top view of the needle assembly of FIG. 1 with the needle shield in a retracted position;

FIG. 8 is a top view of the needle assembly of FIG. 4 with the needle shield in a fully shielding position;

FIG. 9 is a top cross-sectional view of the needle assembly of FIG. 1 with the needle shield in a retracted position;

FIG. 10 is a top cross-sectional view of the needle assembly of FIG. 4 with the needle shield in a fully shielding position;

FIG. 11 is a side cross-sectional view of the needle shield in use with a needle in a retracted position;

FIG. 12 is a side cross-sectional view of the needle shield in use with a needle in a fully shielding position; and

FIG. 13 is a perspective view of a needle clip for use in one embodiment of the present invention.

### DETAILED DESCRIPTION

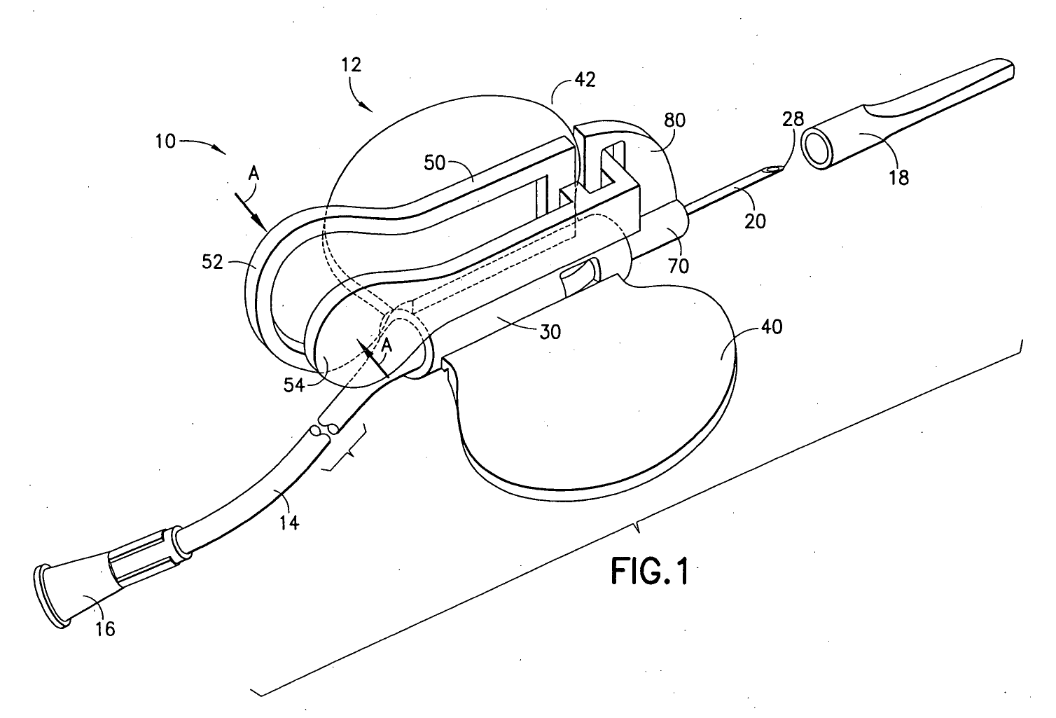

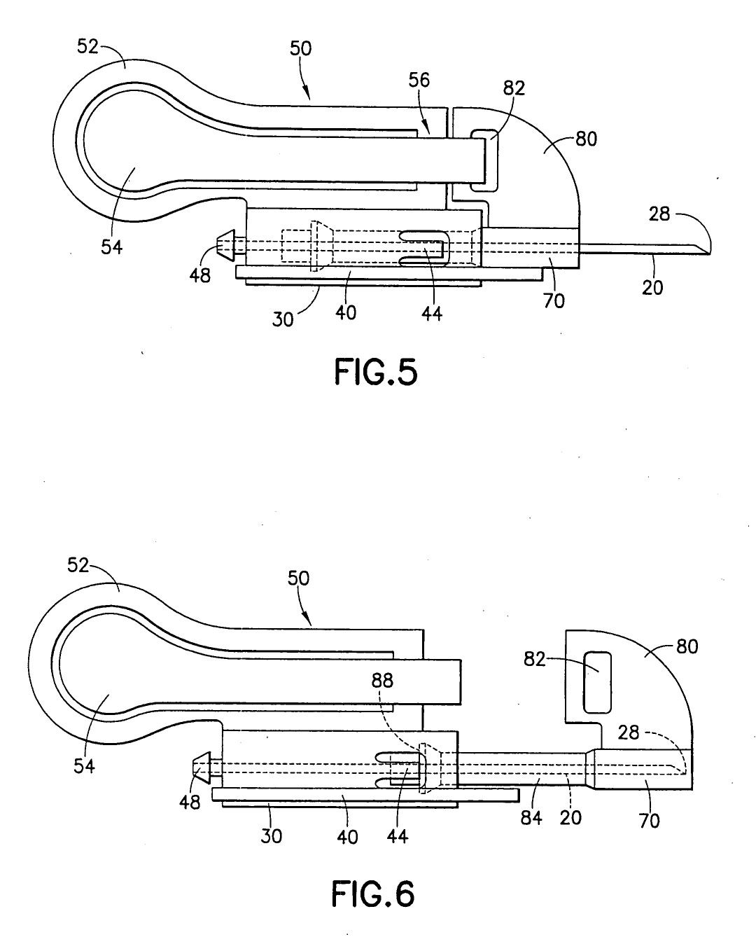

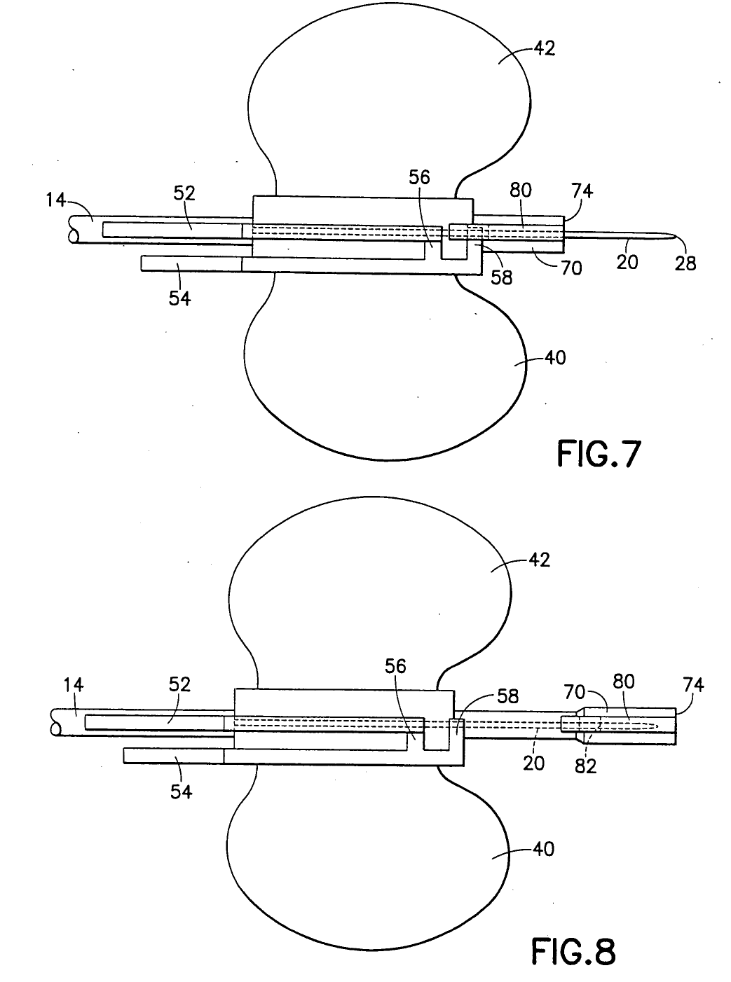

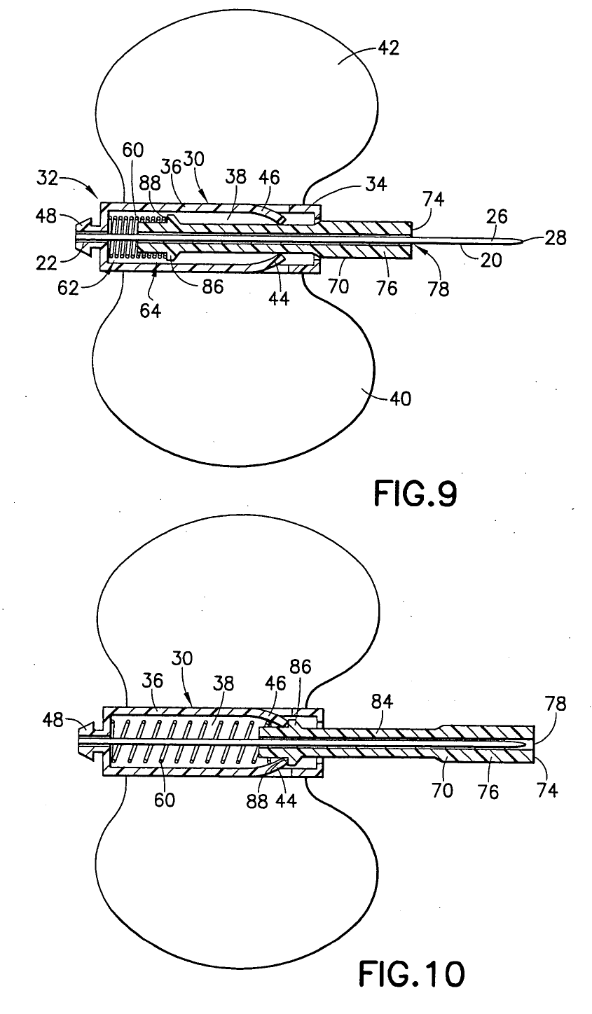

FIGS. 1-10 generally illustrate a medical device according to the present invention. The medical device of the present invention generally includes a shieldable safety needle system adapted to enclose or surround a used needle cannula at the end of a medical procedure, such as a blood collection procedure. The medical device may be used as part of a fluid collection set used to collect blood or other fluids from the body of a human or animal. While described herein in terms of a blood collection set 10, it is noted that the medical device of the present invention including the safety needle system may be used with any medical device incorporating a needle, such as a syringe system, a double-ended phlebotomy needle, or the like. Preferably, the medical device includes a portion that is automatically biased to a safety, needle-enclosing position during normal use by a user of the medical device resulting in a passive activation of the safety features of the assembly, as discussed in detail hereinafter.

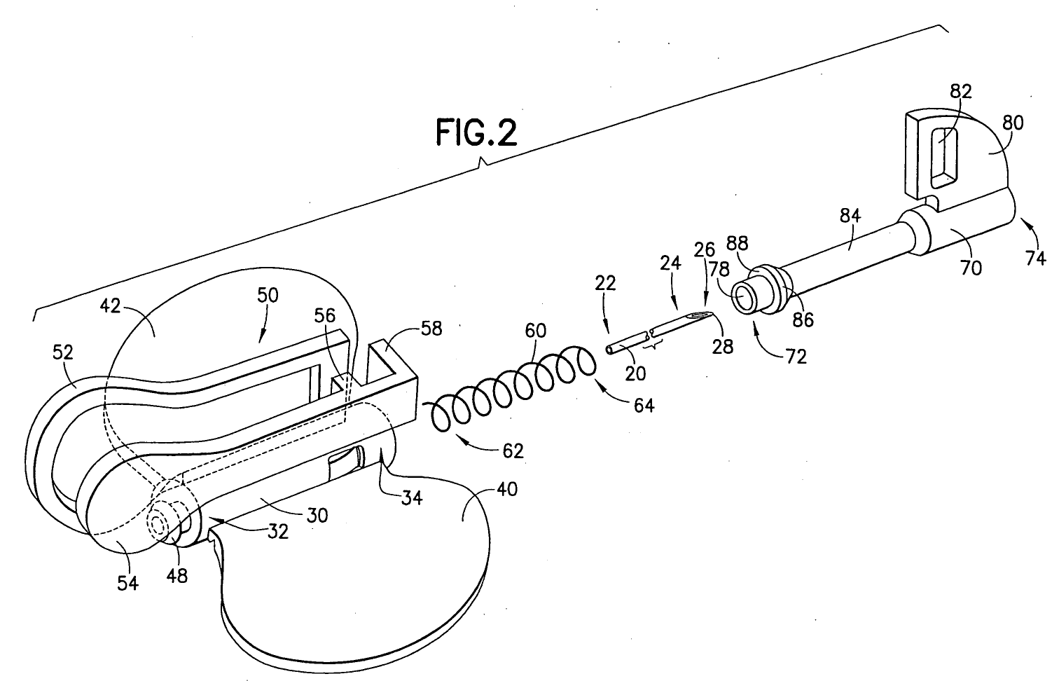

Referring to FIGS. 1 and 2, a first embodiment of the medical device is shown in the form of blood collection set 10. Blood collection set 10 generally includes a shieldable needle device 12, a flexible tube 14 extending from needle device 12, a fixture 16 mounted to tube 14, and a packaging cover 18 removably mounted to portions of needle device 12 opposite tube 14, such as through a frictional engagement. Shieldable needle device 12 of blood collection set 10 is shown in detail in FIGS. 2-10, and generally includes a needle cannula 20, a hub housing 30, a shield housing 70, a biasing element in the form of spring 60 biasing the shield housing away from the hub housing, and a latch mechanism 50 for maintaining the shield housing 70 in a biased state adjacent hub housing 30. The hub housing 30 is adapted for connection to a receptacle (not shown) of, for example, a blood collection set, by way of fixture 16 extending from hub housing 30 through flexible tube 14 by means and procedures known in the art.

Needle cannula 20 includes a proximal end 22 and an opposing distal end 24, with lumen 26 extending through needle cannula 20 from proximal end 22 to distal end 24. Distal end 24 of needle cannula 20 is beveled to define a sharp puncture tip 28, such as an intravenous puncture tip. Puncture tip 28 is provided for insertion into a patient's blood vessel, such as a vein, and is therefore designed to provide ease of insertion and minimal discomfort during venipuncture. Packaging cover 18 is positioned over the distal end 24 of the needle cannula 20 as a removable protective cover to prevent accidental needle-stick wounds prior to using the blood collection set 10 in a medical procedure and to protect the puncture tip 28 prior to use.

Needle assembly 12 further includes hub housing 30. Hub housing 30 is a unitary structure, desirably molded from a thermoplastic material. Hub housing 30 includes a first, open proximal end 32, a second, open distal end 34, and is defined by a rigid tubular wall 36 extending from proximal end 32 to distal end 34. Tubular wall 36 is characterized by an internal passage 38 extending therethrough from proximal end 32 to distal end 34 of hub housing 30. Hub housing 30 includes a nub 48 extending from proximal end 32 thereof, which provides structure for connection of flexible tube 14 thereto. Alternatively, hub housing 30 may incorporate structure for attachment to or engagement with an alternate medical device. For example, hub housing 30 may include a luer fitting, such as a female tapering surface at the proximal end thereof for engagement with a male tapering surface of a separate medical device, such as a syringe barrel.

Hub housing 30 further includes a pair of stabilizers in the form of flexible wings 40 and 42 extending laterally from tubular wall 36 at opposing sides thereof, desirably forming a planar wing structure. Wings 40 and 42 provide hub housing 30, and needle assembly 12, as a butterfly-type wing assembly, for assistance in positioning and placement of needle assembly 12 during a blood collection procedure, and provide a surface for securing needle device 12 to the skin of a patient, such as through taping wings 40 and 42 to the patient's skin. Wings 40 and 42 may be integrally formed with hub housing 30, or may be a separate component or components affixed or adhered to hub housing 30. Wings 40 and 42 may be constructed of a flexible material such that at least one, and preferably both, of the wings 40, 42 may be bent toward each other and brought together between the fingers of the user to assist in positioning and placing the needle device 12 during venipuncture.

The tubular wall 36 of hub housing 30 includes a pair of cutaway portions adjacent distal end 34 in the form of flexible fingers 44 and 46. Flexible fingers 44, 46 are cutaway sections of tubular wall 36 and are biased radially inwardly of tubular wall 36 into internal passage 38. As stated, the tubular wall 36 of hub housing 30 is molded of a thermoplastic material, thereby making the flexible fingers 44 and 46 inherently flexible. Flexible fingers 44, 46 provide needle assembly 12 with a locking mechanism for preventing re-exposure of puncture tip 28 of needle cannula 20 from shield housing 70, as will be described in more detail herein.

Needle cannula 20 is positioned within internal passage 38 of hub housing 30, and extends from distal end 34 of hub housing 30. Desirably, needle cannula 20 and hub housing 30 are separate parts which are fixedly attached and secured through an appropriate medical grade adhesive or the like. More particularly, the proximal end 22 of needle cannula 20 is received within the internal passage 38 of hub housing 30, with the distal end 24 of needle cannula 20 projecting outward from the distal end 34 of the hub housing 30. The internal passage 38 communicates with the lumen 26 defined in the needle cannula 20 to enable fluid, such as blood, to pass through the needle device 12 and to the flexible tube 14 connecting the needle device 12 to the blood collection receptacle. The needle cannula 20 is preferably secured adjacent the proximal end 32 of hub housing 30 through the use of an appropriate medical grade adhesive, mechanical means, or the like.

A latch mechanism 50 extends dorsally from the top surface of hub housing 30. Latch mechanism 50 includes a first member and a second member 54 extending from a portion of the first member 52. First member 52 and second member 54 form generally elongated finger tabs which extending axially toward the proximal position, i.e., toward proximal end 32 of hub housing 30. Second member 54 includes a forward or distally extending portion with a hook 58 at a distal end thereof, and extends from first member 52 through a lever arm which creates a fulcrum 56. As such, first member 52 and second member 54 can be squeezed or pinched toward each other about fulcrum 56, which causes hook 58 at the distal portion of second member 54 to pivot outwardly.

As noted, wings 40 and 42 extend from opposing lateral sides of hub housing 30, generally in a planar fashion. Latch mechanism 50 extends dorsally from the top surface of housing 30 and generally bisects the plane defining wings 40 and 42, such that wings 40, 42 are generally normal to latch mechanism 50. Moreover, first member 52 and second member 54 of latch mechanism 50 extending in a generally proximal direction such that bending together of wings 40 and 42 in a dorsal manner does not cause first member 52 and second member 54 to be squeezed or pinched together due to the positioning of the finger tabs of first member 52 and second member 54 and of the lever arm or fulcrum 56, as will be discussed further with respect to use of the device.

Needle device 12 further includes shield housing 70. The shield housing 70 extends between a first, open proximal end 72 and a second, open distal end 74, and is defined by a generally hollow tubular wall 76 having an internal passage 78. Shield housing 70 is preferably a unitary structure, which is desirably molded from a thermoplastic material. The internal passage 78 is sized to coaxially receive needle cannula 20, with shield housing 70 axially slidable thereabout. As such, shield housing 70 is movable along needle cannula 20 between a first proximal or retracted position adjacent hub housing 30, and a second distal position encompassing and shielding puncture tip 28, as will be described in more detail herein.

The proximal end 72 of shield housing 70 is sized to be received within the distal end 34 of hub housing 30 and extend within internal passage 38 thereof. Accordingly, tubular wall 76 of shield housing 70 may include a portion of reduced diameter at stepped down portion 84, which extends toward the proximal end 72 of shield housing 70. This stepped down portion 84 slidably cooperates within the internal passage 38 of hub housing 30. Shield housing 70 may further include a neck 86 extending radially about the stepped down portion 84, forming a shoulder 88 facing the proximal end 72 of shield housing 70 for interference engagement with flexible fingers 44, 46 to providing a locking mechanism, as will be discussed in more detail herein.

Shield housing 70 is generally movable between a first or retracted position adjacent hub housing 30 with at least a portion of shield housing 70, such as proximal end 32 thereof, coaxially contained within internal passage 38 of hub housing 30, and a second, extended or needle-shielding position, wherein the needle cannula 20 is enclosed or disposed within shield housing 70 fully covering the needle cannula 20 and, in particular, the puncture tip 28. The first or retracted position of the shield housing 70 is shown in FIGS. 1, 5, 7 and 9 and the second or extended position of the shield housing 70 is shown in FIGS. 4, 6, 8 and 10. In the first retracted position, the distal end 24 of needle cannula 20 projects outward from the shield housing 70 for use in a fluid collection procedure.

A latch element 80 extends dorsally from the top surface of shield housing 70, forming a dorsal wing-like structure. Latch element 80 includes a recessed portion within the surface thereof, or an opening such as opening 82 extending through the surface thereof. Opening 82 of latch element 80 is provided for establishing releasable engagement with hook 58 of latch mechanism 50 of hub housing 30. As such, the engagement between hook 58 of latch mechanism 50 within recess or opening 82 of latch element 80 establishes a dorsally extending engagement mechanism for releasably retaining shield housing 70 in the proximal or retracted position adjacent hub housing 30.

The needle device 12 preferably further includes a biasing member such as compression spring 60 extending between the hub housing 30 and the shield housing 70. Spring 60 is adapted to act on the shield housing 70 to bias the shield housing 70 from the first retracted position adjacent hub housing 30 toward the second shielding position in which shield housing 70 encompasses puncture tip 28 of needle cannula 20. The biasing member or spring 60 is disposed within internal passage 38 of hub housing 30 about needle cannula 20, and extends between the internal surface within internal passage 38 at the proximal end 32 of hub housing 30 and shoulder 88 of neck 86 extending circumferentially about the proximal end of shield housing 70 extending within hub housing 30. Spring 60 is maintained in a compressed state by engagement of the hook 58 of the second member 54 of latch mechanism 50 within the recess or opening 82 in the latch element 80 of shield housing 70. If desired, the proximal end 62 of the spring 60 may be secured to the proximal end 32 of hub housing 30 by a suitable medical grade adhesive known in the art or by a fixed mechanical connection. Likewise, the distal end 64 of the spring 60 may be adhesively or mechanically secured to the shield housing 70, such as at shoulder 88. Once the spring 60 is released by disengaging the hook 58 from the corresponding opening 82, the spring 60 exerts an axial expansion force against the shield housing 70, and in particular, against the proximal end 72 or shoulder 88 of shield housing 70, relative to the hub housing 30. The shield housing 70 is urged by the spring 60 to the second, needle-shielding position, wherein the shield housing 70 covers the needle cannula 20 (i.e., FIG. 4). The spring 60 also substantially prevents the shield housing 70 from moving proximally back toward the first position within hub housing 30 by providing a counter-acting biasing force against such a movement.

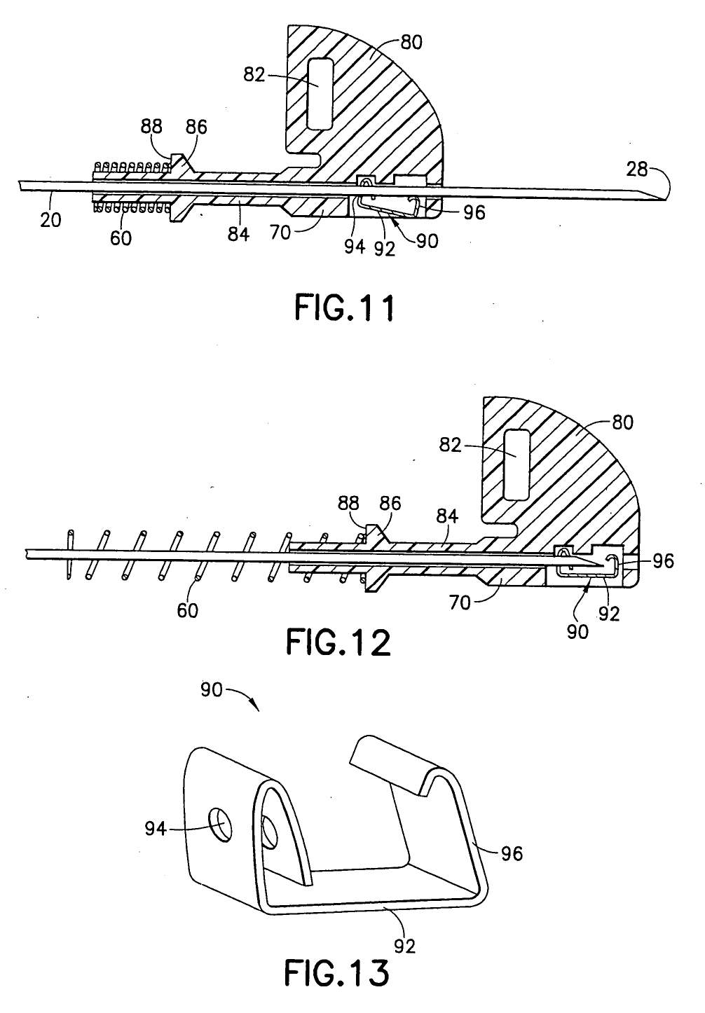

In one embodiment, shield housing 70 may further include a protective clip 90 for locking out puncture tip 28 of needle cannula 20 and preventing re-exposure thereof. As best seen in FIGS. 11-13, clip 90 may be unitarily stamped and formed from a resiliently deflectable metallic material. Clip 90 includes a planar spring leg 92 extending between a proximal end and an opposed distal end. A mounting aperture 94 extends through clip 90 at a location near the proximal end. A lock out leg 96 extends angularly from the distal end of clip 90, and is bent back toward the proximal end of clip 90. The bends in lock out leg 96 enable secure protective engagement with puncture tip 28 of needle cannula 20 and further enable smooth axial sliding movement of shield housing 70 along needle cannula 20, as described in further detail herein.

In use, blood collection set 10 is provided with needle device 12 assembled and including flexible tube 14 extending from needle device 12 and connected to fixture 16. Blood collection set 10 is preferably packaged with the shield housing 70 in the first retracted position, as shown, for example, in FIG. 1. After removing blood collection set 10 from its package, it can be assembled with other appropriate medical equipment for use. For example, a non-patient needle assembly and a needle holder may be connected to blood collection set 10 through fixture 16.

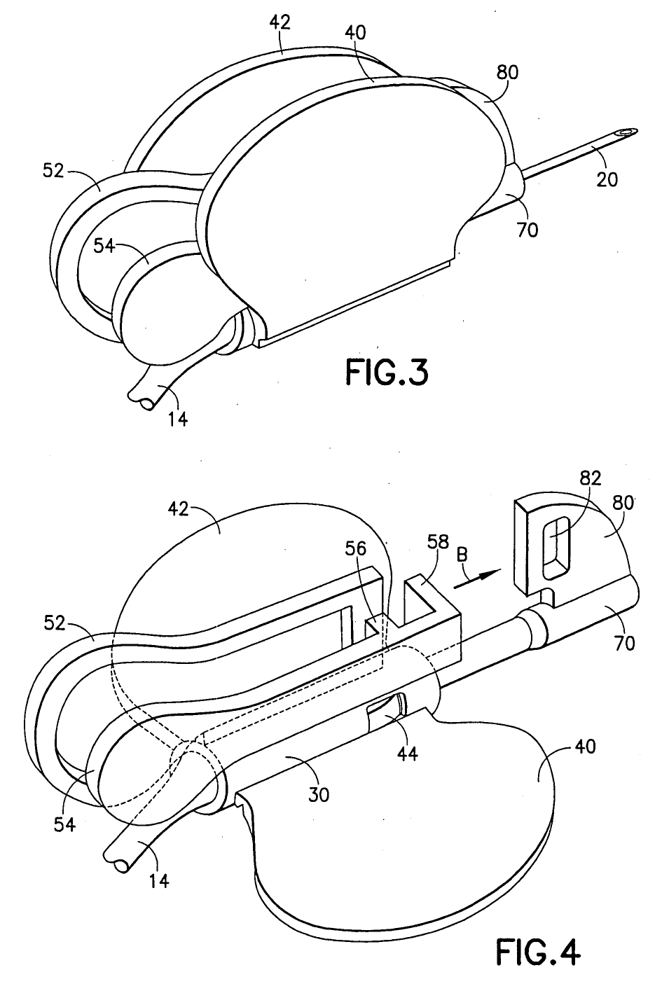

To prepare for use of blood collection set 10, the user grasps wings 40 and 42 at opposing lateral sides of hub housing 30 between a thumb and forefinger, and bends wings 40 and 42 dorsally toward each other, with latch mechanism 50 extending therebetween, as shown in FIG. 3. Packaging cover 18 is then grasped and urged distally to disengage from needle cannula 20 and/or shield housing 70, thereby exposing puncture tip 28 of needle cannula 20.

The overall design and profile of latch mechanism 50 is configured such that bending of the wings 40 and 42 to the dorsal position for positioning and placement does not cause activation of the latch mechanism 50. In other words, bending of wings 40 and 42 together does not cause pinching movement of the finger tabs of first member 52 and second member 54 relative to each other, which would disengage the hook 58 from interference engagement with opening 82. This may be accomplished by maintaining the lever arm or fulcrum 56 at a position directly adjacent the point of contact between bent wings 40 and 42. As such, shield housing 70 is maintained in the first retracted position with latch mechanism 50 unactivated during bending of wings 40 and 42 for positioning and placement of needle device 12. Desirably, wings 40 and 42 are of sufficient length to extend dorsally beyond latch mechanism 50 when wings 40 and 42 are bent together, thereby providing further assurance that latch mechanism 50 will not be activated to release shield housing 70 during positioning and placement.

After packaging cover 18 is removed, the medical practitioner can then urge puncture tip 28 at distal end 24 of needle cannula 20 into a targeted blood vessel of a patient with wings 40 and 42 bent inwardly toward each other between the user's fingers to act as a structure for guided placement, as is known in the art. After the targeted blood vessel has been accessed, the medical practitioner can release the grip on wings 40 and 42, and wings 40 and 42 can thereafter be taped to the patient's skin to prevent movement of the needle device 12 during use.

Upon completion of the procedure, such as when all desired samples have been drawn, needle cannula 20 can be withdrawn from the patient. As opposed to conventional techniques for withdrawing wingsets by again bending the wings together in a dorsal manner, withdrawal of needle device 12 is preferably accomplished by grasping latch mechanism 50, that is by grabbing the finger tabs of first member 52 and second member 54 between a user's thumb and forefinger. This grabbing causes first member 52 and second member 54 to move toward each other in a direction of arrows A in FIG. 1, with fulcrum 56 acting as a pivot point for movement thereof. This movement toward each other in turn causes the distal end portion of second member 54 to pivot outwardly, which causes hook 58 to disengage from interference engagement within opening 82 of latch element 80 of shield housing 70. Once the interference engagement between hook 58 and opening 82 is released, the bias from spring 60 causes shield housing 70 to be propelled distally in a direction of arrow B in FIG. 4, away from hub housing 30 along needle cannula 20. At this point, the puncture tip 28 of needle cannula 20 may still be positioned within the patient. As such, shield housing 70 is propelled forward to a position in which it contacts the patient's skin. The user can then remove the needle device 12 from the patient, during which the shield housing 70 continues to move forward under the bias of the spring 60, until the needle cannula 20 is fully withdrawn from the patient, at which point the shield housing 70 extends to the fully shielding position completely encompassing puncture tip 28. After shield housing 70 is moved along needle cannula 20 past the puncture tip 28, lockout leg 96 of clip 90 will pass distally beyond puncture tip 28 of needle cannula 20. The inherent resiliency of spring leg 92 of clip 90 will urge lockout leg 96 over puncture tip 28 of needle cannula 20. Thus, a return movement of shield housing 70 is prevented through clip 90 acting as a locking mechanism. Hence, puncture tip 28 of needle cannula 20 is safely shielded. Blood collection set 10 may then be appropriately discarded.

In addition to or instead of the locking mechanism established through clip 90, a further locking mechanism may be provided through the interference interaction between flexible fingers 44, 46 and shoulder 88 of neck 86 on shield housing 70. More particularly, as spring 60 propels shield housing 70 distally in the direction of arrow B, neck 86 of shield housing 70 passes beyond flexible fingers 44, 46, which are biased radially inwardly against the outer surface of shield housing 70. Once neck 86 passes beyond flexible fingers 44, 46, the flexible fingers 44, 46 abut against shoulder 88, thereby preventing shield housing 70 from movement backward in the opposite direction, as seen in FIG. 10. As such, shield housing 70 is locked in the shielding position encompassing puncture tip 28. It will be appreciated that the location of the flexible fingers and corresponding shoulder may be reversed in accordance with the present invention. In addition, neck 86 of shield housing 70 may be trapped by the distal end of hub housing 30 to prevent movement beyond hub housing 30, such as through a return portion within the distal end 34 of hub housing 30 which prevents the passage of neck 86 therebeyond.

Alternatively, if desired, activation of latch mechanism 50 to release shield housing 70 can be accomplished immediately after needle cannula 20 is placed within the patient's blood vessel, as opposed to waiting for withdrawal of needle device 12 from the patient.

Alternatively, activation of latch mechanism 50 may be delayed until after needle cannula 20 is fully removed from the patient. For example, wings 40 and 42 may be bent together in a dorsal manner to create a structure for holding needle device 12 and withdrawing needle cannula 20 from the patient. After the needle cannula 20 is withdrawn in this manner, activation of latch mechanism 50 can be accomplished to release shield housing 70 and effectively shield the used puncture tip 28. As such, the needle device of the present invention is selectively passive.

The inner surface of the hub housing 30 and the outer surface of the shield housing 70 may interact such that any axial rotation of the hub housing 30 with respect to the shield housing 70 is inhibited during the sliding movement of the shield housing 70 from the first retracted position to the second shielding position. For example, a surface feature may be provided on one of the outer surface of the shield housing 70 or the inner surface of the hub housing 30, such as a plurality of spline grooves or detents. Alternatively, the hub housing 30 and shield housing 70 may each have a non-circular cross section, for example, with one planar side, desirably a bottom flat surface, to prevent relative rotation between these elements.

The needle device of the present invention is particularly innovative since it involves a passive activation of the safety shielding feature through the ergonomic layout of the device. In particular, when in the retracted position, the shield housing interengages with the hub housing to form a grippable dorsal housing structure with the hub housing forming a proximal housing structure and the shield housing forming a distal housing structure, and incorporating a latch mechanism extending dorsally therebetween. Moreover, the shield housing is automatically activated to shield the needle cannula during the typical steps involved in a venipuncture procedure. For example, the device is easily insertable within a patient using a normal technique with bent wings forming a dorsal structure for guiding, positioning, and placement of the device. The shield housing can remain retracted during the blood sampling procedure, thereby preventing the shield housing from interfering with the user during the sampling procedure, and preventing the patient from being startled by activation of the shield against the patient's skin prior to the sampling procedure. Moreover, activation is easily accomplished during a typical procedure for removing the needle device from the patient, i.e., by grasping the device in a dorsal manner, through the latch mechanism extending dorsally from the device. This grasping automatically passively activates the device during removal, thereby avoiding any active step for shielding of the assembly by the user, and preventing any unnecessary exposure to the used needle cannula. The selectively passive activation affords a greater degree of safety to the user with minimal or no change in technique, as compared with conventional safety needle mechanisms.

While the medical device of the present invention is described in terms of several preferred embodiments for use in connection with bodily fluid collection systems and like devices, the present invention may take many different forms. The preferred embodiments shown in the drawings and described hereinabove in detail are to be considered as exemplary of the principles of the invention and are not intended to limit the invention to the embodiments illustrated. Various other embodiments will be apparent to and readily made by those skilled in the art without departing from the scope and spirit of the invention. The scope of the present invention will be measured by the appended claims and their equivalents.

1. A safety needle system, comprising:
a hub housing having a first end and an opposed second open end with a passageway extending therebetween;
an elongated shield housing having a first open end and an opposed second open end with a passageway extending therebetween;
a needle cannula having a distal puncture tip, the needle cannula extending from the hub housing with at least a portion of the needle cannula extending through the passageway of the shield housing;
a biasing member acting on the shield housing to bias the shield housing from a first biased position adjacent the hub housing toward a second position covering the distal puncture tip of the needle cannula; and
an engagement mechanism extending dorsally from the hub housing, the engagement mechanism in releasable engagement with a portion of the shield housing for releasably retaining the shield housing in the first biased position; wherein engagement of the engagement mechanism with the shield housing maintains the shield housing in the first biased position adjacent the hub housing, and wherein activation of the engagement mechanism releases the engagement mechanism from engagement with a portion of the shield housing, thereby releasing the shield housing from the biased position and permitting the biasing member to cause the shield housing to move toward the second position.

2. The safety needle system as in claim 1, wherein the engagement mechanism comprises a first member extending dorsally from the hub housing, and a second member extending from a portion of the first member and in engagement with a portion of the shield housing, the second member moveable with respect to the first member so as to release from engagement with the shield housing.

3. The safety needle system as in claim 2, wherein the shield housing includes a latch element extending dorsally from the shield housing, the latch element including a recess or opening therein for engagement with the engagement mechanism of the hub housing.

4. The safety needle system as in claim 3, wherein the second member of the engagement mechanism includes a hook element for engagement with the recess or opening of the latch element.

5. The safety needle system as in claim 4, wherein the second member of the engagement mechanism is connected to the first member of the release arrangement through a fulcrum.

6. The safety needle system as in claim 5, wherein the first and second members of the engagement mechanism include corresponding surfaces for movement toward each other about said fulcrum, thereby causing movement of said hook element out of engagement from said recess or opening of said latch element.

7. The safety needle system as in claim 1, wherein said hub housing further comprises structure for attachment to a medical device.

8. The safety needle system as in claim 1, wherein the biasing member comprises a compression spring.

9. The safety needle system as in claim 1, wherein a portion of the shield housing extends within the passageway of the hub housing.

10. The safety needle system as in claim 9, wherein the hub housing further comprises at least one flexible cut out portion along a wall thereof, said flexible cut out portion biased inwardly toward said passageway of said hub housing.

11. The safety needle system as in claim 10, wherein said flexible cut out portion is adapted for engagement with a portion of said shield housing when said shield housing is in said second position to prevent a return movement of said shield housing to said first position.

12. The safety needle system as in claim 1, further comprising a pair of flexible wings extending from opposing lateral sides of said hub housing.

13. The safety needle assembly as in claim 12, wherein the engagement mechanism extending dorsally from the hub housing bisects the flexible wings extending from opposing lateral sides of the hub housing, and wherein bending of the flexible wings toward a dorsal position does not cause activation of the engagement mechanism.

14. A method for passively activating a safety needle system comprising:
providing a safety needle system comprising a hub housing including a needle cannula extending from a distal end of said hub housing toward a distal puncture tip and a shield housing covering at least a portion of said needle cannula adjacent said hub housing, said safety needle system further including a biasing element for biasing said shield housing toward a shielding position fully covering said distal puncture tip of said needle cannula and a latch mechanism extending dorsally from said safety needle system for maintaining said shield housing in a biased state adjacent said hub housing;
inserting said safety needle system into a patient;
withdrawing said safety needle system from said patient by grasping said latch mechanism, thereby releasing said shield housing from said biased state and causing said shield housing to extend toward said shielding position.

15. The method of claim 14, wherein said hub housing further includes a pair of flexible wings extending from opposing lateral sides thereof, and wherein said inserting step comprises bending said flexible wings to a dorsal position for guiding said needle cannula into the patient.

16. The method of claim 15, wherein bending of said flexible wings to a dorsal position does not cause said latch mechanism to release said shield housing from said biased state.

17. The method of claim 14, wherein the latch mechanism comprises a first member extending dorsally from said hub housing and a second member connected to said first element through a fulcrum, wherein said second element and said shield housing include corresponding structure for latching engagement therebetween, and wherein said withdrawing step comprises grasping said first member and said second member, thereby pivoting said second member about said fulcrum causing release of said corresponding structure from latching engagement therebetween, thereby causing said shield housing to extend toward said shielding position due to said biasing element.

18. The method of claim 17, wherein said corresponding structure for latching engagement comprises a hook element extending from said second member in engagement with a recess or opening within a dorsally extending portion of said shield housing.

19. The method of claim 14, wherein the shield housing is prevented from reexposing the distal puncture tip of the needle cannula once the shield housing has been extended to a fully shielding position.

20. A safety needle system, comprising:
a grippable dorsal housing comprising distal and proximal dorsal housing portions, said proximal dorsal housing portion supporting a needle and said distal dorsal housing portion extendable in an axial direction with respect to said needle from a first position adjacent said proximal housing portion to a second position covering a distal tip of said needle;
a planar wing structure integral to at least one of said proximal and distal dorsal housing portions, said planar wing structure extending generally normal to said grippable dorsal housing;
a biasing element extending between said distal and proximal dorsal housing portions and biasing said dorsal housing portion toward said second position; and
a release element for selectively retaining said distal dorsal housing portion in said first position adjacent said first housing portion against the bias of said biasing element.

21. A safety needle system as in claim 20, wherein said release element comprises a release latch and a receiving element for receiving said latch.

22. A safety needle system as in claim 21, wherein said release latch is integral with said proximal dorsal housing portion.

23. A safety needle system as in claim 21, wherein said receiving element comprises a recess or opening within said distal dorsal housing portion.
